(19) United States
(12) Patent Application Publication
(10) Pub. No.: US 2008/0286335 A1
(43) Pub. Date: Nov. 20, 2008

(54) Enhancement Of The Calcium Sensing Receptor

(75) Inventors: Nicholas Cotton, Westborough, MA (US); Catherine Booth, Hingham (GB); James Huckle, West Rounton (GB); Graeme Howling, Roundhay (GB)

Correspondence Address: NORMAN F. HAINER, JR.;SMITH & NEPHEW, INC., 150 MINUTEMAN ROAD, ANDOVER, MA, 01801, US

(73) Assignee: Smith & Nephew, Inc., Andover, MA (US)

(21) Appl. No.: 12/119,045

(22) Filed: May 12, 2008

Related U.S. Application Data
(60) Provisional application No. 60/917,134, filed on May 10, 2007.

(51) Int. Cl. A61F 2/04; A61K 33/06; A61P 19/08; A61K 33/10

(52) U.S. Cl. 424/426; 424/682; 424/687; 424/423

(57) ABSTRACT

The disclosure relates to medical devices capable of enhancing ossification in a subject. More specifically, the disclosure relates to the use of devices comprising a releasable calcium sensing receptor agonist and a releasable calcium salt.

### CROSS REFERENCE TO RELATED APPLICATIONS

This application claims priority to U.S. Patent Application No. 60/917,134 filed on May 10, 2007, the disclosure of which is incorporated herein by reference in its entirety.

### BACKGROUND

1. Field of Technology

The present disclosure relates to medical devices capable of enhancing ossification in a subject. More specifically, the disclosure relates to the use of devices comprising at least one releasable calcium sensing receptor agonist and at least one releasable calcium salt.

2. Related Art

Extracellular Ca²⁺ controls a number of fundamental processes within the body such as blood clotting, nerve and muscle excitability and bone formation. As a result the concentration of extracellular Ca²⁺ is under tight homeostatic regulation.

Bone formation in response to a localised delivery of soluble Ca²⁺ has been studied. The study involved the doping of alumina tubes with various elements, including Ca²⁺, which were then implanted into the femoral meduallary canals of female rats. In contrast to the non-doped alumina tubes, Ca²⁺-doped tubes were found to exhibit enhanced osteogenesis in the form of advancing tissue fronts of lamellar bone. This study is further described in Pabbruwe et al., Biomaterials, vol. 25, pp. 4901-4910 (2004), the disclosure of which is incorporated herein by reference in its entirety.

In vivo studies involving isolated rat osteoclasts have shown that osteoclast activity is down-regulated by Ca²⁺ released into the environment by resorbing osteoclasts. Hence, a net increase in bone formation is observed. These studies are further described in Datta et al., Biosci. Rep., vol. 9, pp. 747-751 (1989) and Zaidi et al., J. Cell. Physiol., vol. 149, pp. 422-427, the disclosures of which are incorporated herein by reference in their entireties.

There is also evidence linking extracellular calcium levels with expression of bone morphogenic protein (BMP) molecules, specifically BMP-2 and BMP-4), growth factors known for their ability to induce the formation of bone and cartilage. This evidence is further described in Nakade et al., J. Bone Mineral. Metab, vol. 19, pp. 13-19, the disclosure of which is incorporated herein by reference in its entirety.

Based upon the osteoconductive properties of Ca²⁺, a range of osteoconductive materials have been developed for use in the field of bone and cartilage repair. Such materials include, for example, calcium phosphates, collagen/CaP composites, and calcium sulfates.

Smith & Nephew Inc. developed an osteoconductive interference screw, which promotes bone regrowth after anterior cruciate ligament (ACL) reconstruction. The screw is molded from a blend of bioabsorbable polymer and calcium carbonate, referred to as PLC. The screw is used to secure a graft in ACL reconstruction. Over the course of the next several months, the screw is resorbed by the body, and the calcium carbonate within the screw stimulates the natural process of bone formation in its place. This new bone fills the tunnel where a surgeon placed the graft, and promotes ossification of the graft. This screw is more fully described in United States Patent Application Publication US 2006/0120994, the disclosure of which is incorporated herein by reference in its entirety.

The calcium sensing receptor (CaSR), a member of the seven-transmembrane receptor super family, is believed to be the major mechanism by which systemic levels of Ca²⁺ in the body are detected and controlled. Although initially discovered on the surface of parathyroid cells, its expression has been demonstrated on a range of cells including osteoclasts in bone, the juxtaglomerular and proximal tubule cells in the kidney, keratinocytes in the epidermis, parafollicular cells in the thyroid, intestinal cells, and the trophoblast in the placenta. This expression is more fully described in U.S. Pat. No. 5,858,684, the disclosure of which is incorporated by reference in its entirety. Fluctuations in systemic calcium are known to influence bone formation indirectly by inducing changes in parathyroid hormone (PTH) and 1,25(OH)2-vitamin D3, however the expression on osteoclasts and osteoblast would also suggest a possible direct affect on bone cell function and bone formation.

CaSR agonists are ligands which mimic or potentiate the activity of extracellular Ca²⁺ at the CaSR. These small molecules, also referred to as calcimimetic agents, increase the sensitivity of the CaSR receptor to Ca²⁺ The agonists have been classified into type I agonists which have effect on their own regardless of whether Ca²⁺ is present or not and type II agonists which change the affinity of Ca²⁺ through allosteric actions and potentiate the action of the receptor's polycationic agonists.

It is known in the art to use agonists of the CaSR in order to treat a number of diseases and disorders, see for example International Application Publication WO 01/83546, the disclosure of which is incorporated herein by reference in its entirety. Due to the fact that the type II agonists increase the sensitivity of the CaSR to extracellular Ca²⁺, one of the problems associated with their use is their dependency on sufficient extracellular levels of Ca²⁺. For example, in hypocalcaemic subjects, diagnosed by serum calcium <8.2 mg/dL (2.05 mmol/L) or ionized calcium <4.4 mg/dL (1.1 mmol/L), the use of a type II agonist may have a significantly reduced efficacy.

The present disclosure provides a means of enhancing the sensitivity of the CaSR to exogenous Ca²⁺, by providing a releasable calcium salt in combination with a releasable calcium sensing receptor (CaSR) agonist.

The present disclosure provides a means of enhancing the efficacy of the CaSR agonists, by providing a releasable calcium sensing receptor (CaSR) agonist in combination with releasable calcium salt.

### SUMMARY

According to an aspect of the disclosure there is provided a device comprising a releasable calcium sensing receptor (CaSR) agonist and a releasable calcium salt.

Suitable examples of CaSR agonists are outlined below. This list is by no means exhaustive and any molecule or agent having agonistic or calcimimetic activity is considered to be within the scope of the disclosure. For example, any agent or molecule that is positively charged at physiological pH has the potential to be a calcimimetic.

The calcimimetic can be a natural or synthetic molecule, agent, or compound. The disclosure also covers any structural variant or analogue of the calcimimetics outlined. Calcimimetics include a variety of inorganic and organic polyvalent cations. Examples of suitable divalent cations include: Ca²⁺, Mg²⁺, Zn²⁺ and Sr²⁺. An example of a suitable trivalent cation is Gd³⁺. The inorganic and organic polyvalent cations are examples of type I calcimimetics.

In an embodiment of the disclosure, the calcimimetic is an inorganic and organic polyvalent cation.

Calcimimetics also include many structurally diverse organic compounds which share the common property of possessing a net positive charge at physiological pH.

Organic compounds which act as calcimimetics include, for example, polyamines, aminoglycoside antibiotics, phenylalkylamines, and a number of amino acids.

Polyamines are organic compounds having two or more primary amino groups, such as putrescine, cadaverine, spermidine, and spermine, that are growth factors in both eukaryotic and prokaryotic cells. In structure the polyamines represent compounds with cations that are found at regularly-spaced intervals.

In an embodiment of the disclosure, the calcimimetic is a branched or cyclic polyamine. These have been shown to potentially have higher calcimimetic activity than their straight chain analogues.

Another group of calcimimetics are the aminoglycoside antibiotics which are particularly bactericidal against gram negative aerobic bacteria. Aminoglycosides are a group of antibiotics that are effective against certain types of bacteria and include amikacin, gentamicin, kanamycin, neomycin, netilmicin, paromomycin, streptomycin, tobramycin and apramycin.

In an embodiment of the disclosure, the calcimimetic is an aminoglycoside antibiotic.

A further group of calcimimetics are the arylalkyl polyamines which are a class of positively charged products derived from arthropod venoms.

Therefore, in a still further embodiment of the disclosure, the calcimimetic is a natural or synthetic arylalkyl polyamine.

Amino acids have been shown to be type II calcimimetics. For example, in the presence of Ca²⁺ the CaSR can be activated allosterically by L-amino acids in the millilmolar concentration range with a preference for aromatic and small aliphatic L-amino acids.

In embodiments of the disclosure, the calcimimetic is an amino acid or a polyamino acid. A polyamino acid herein refers to polypeptides containing two or more amino acid residues which are positively charged at physiological pH. It is particularly advantageous that the amino acid(s) is an aromatic amino acid or an aliphatic amino acid. Examples of aromatic amino acids include phenylalanine (Phe), tyrosine (Tyr) and tryptophan (Trp). Examples of aliphatic amino acids include glycine (Gly), alanine (Ala), valine (Val), leucine (Leu) and isoleucine (Ile).

The twenty naturally occurring amino acids may all be of the L-(Laevorotatory) form. This is the stereoisomer that rotates plane polarized light to the left. It has been found that calcimimetic effect of an amino acid at the CaSR is more potent when the amino acid is in the L-form rather than the D-form. In embodiments of the disclosure, the amino acid is in the L-steroisomeric form. In further embodiments of the disclosure, the amino acid is provided as a mixture of the L- and D-forms.

In some embodiments of the disclosure, the L-amino acid is L-Phe, its mode of release from the device being such that a therapeutic concentration of L-Phe in the vicinity of the device of between about 0.5 mM and 5 mM is provided, more particularly a therapeutic concentration of between about 1 mM and 3 mM, and even more particularly a therapeutic concentration of between about 1 mM and 2.5 mM.

In other embodiments of the disclosure, the L-amino acid is L-Trp, its mode of release from the device being such that a therapeutic concentration of L-Trp in the vicinity of the device of between about 0.5 mM and 5 mM is provided, more particularly a therapeutic concentration of between about 1 mM and 3 mM, and even more particularly a therapeutic concentration of between about 1 mM and 2.5 mM.

In embodiments of the disclosure, the device comprises two or more different type 1 agonists. For example, the device can comprise two or more different polyvalent cations (e.g Mg²+ and Zn2+). In other examples, the device can comprise a polyvalent cation (e.g Mg²⁺) and an aminoglycoside antibiotic (e.g neomycin).

In further embodiments of the disclosure, it is envisaged that the device comprises two or more different type 2 agonists, for example an aromatic amino acid (e.g L-Phe) and an aliphatic amino acid (e.g L-Gly).

In still further embodiments of the disclosure, the device comprises at least one type 1 agonist and at least one type II agonist.

Suitable Ca²⁺ salts include, but are not limited to, calcium carbonate (CaCo₃) and calcium chloride (CaCl₂), calcium phosphate (CaPO₄), and calcium sulphate (CaSO₄).

In some embodiments of the disclosure, the mode of release of the calcium salt is such that a therapeutic concentration of calcium salt of between about 0.5 mM and 5 mM is provided in the vicinity of the device, more particularly a therapeutic concentration of between about 1 mM and 3 mM, and even more particularly a therapeutic concentration of between about 1 mM and 2.5 mM.

In advantageous embodiments of the disclosure, the releasable L-amino acid is L-Phe and the releasable calcium salt is CaCl₂, wherein the therapeutic concentration of L-Phe in the vicinity of the device is between about 0.5 mM and 5 mM, more particularly between about 1 mM and 3 mM, and even more particularly between about 1 mM and 2.5 mM, and wherein the therapeutic concentration of CaCl₂ in the vicinity of the device is between about 0.5 mM and 5 mM, more particularly between about 1 mM and 3 mM, and even more between about 1 mM and 2.5 mM.

In a particularly advantageous embodiment of the disclosure, the releasable CaSR agonist is L-Phe, providing a therapeutic concentration in the vicinity of the device of about 2.5 mM and the releasable calcium salt is CaCl₂, providing a therapeutic concentration in the vicinity of the device of about 2.5 mM.

In further advantageous embodiments of the disclosure, the releasable L-amino acid is L-Trp and the releasable calcium salt is CaCl₂, wherein the therapeutic concentration of L-Trp in the vicinity of the device is between about 0.5 mM and 5 mM, more particularly between about 1 mM and 3 mM, and even more particularly between about 1 mM and 2.5 mM, and wherein the therapeutic concentration of CaCl₂ in the vicinity of the device is between about 0.5 mM and 5 mM, more particularly between about 1 mM and 3 mM, and even more particularly between about 1 mM and 2.5 mM.

In a particularly advantageous embodiment of the disclosure, the releasable CaSR agonist is L-Trp, providing a therapeutic concentration in the vicinity of the device of about 2.5 mM and the releasable calcium salt is CaCl₂, providing a therapeutic concentration in the vicinity of the device of about 2.5 mM.

The CaSR agonist and the calcium salt are released from the device either in a time-release, delayed or sustained release, or pulsed release manner. Many types of delivery systems are available and known to those of ordinary skill in the art.

The CaSR agonist and the calcium salt can be released in a simultaneous or sequential manner from the device. For example, it may be advantageous to release the CaSR agonist prior to the release of the calcium salt in order to “prime” the CaSR.

The device is envisaged to be any device which is capable of implantation into a site within a subject and wherein the release of the at least one CaSR agonist and the at least one calcium salt would be beneficial to the subject.

The subject can be any human or non-human animal, such as a companion animal or a farm animal.

The device can be a medical device which is defined as any instrument, apparatus, appliance, material, or other article, whether used alone or in combination, including the software necessary for its proper application intended by the manufacturer to be used for human beings or other animals for the purpose of (i) diagnosis, prevention, monitoring, treatment or alleviation of disease, (ii) diagnosis, monitoring, treatment, alleviation of or compensation for an injury or handicap or (iii) investigation, replacement or modification of the anatomy or of a physiological process, and which does not achieve its principal intended action in or on the human body by pharmacological, immunological or metabolic means, but which may be assisted in its function by such means.

An example of a medical device is an implant that can be provided subcutaneously and which upon controlled release of the at least one calcium agonist and at least one calcium salt has a beneficial therapeutic effect on, for example, metabolic bone diseases such as osteoporosis, osteomalacia, rickets; hypercalcaemia, hypocalcaemia and hyperparathyroidism.

The device can be a surgical device or a dental device. Such devices are designed to perform a therapeutic or correction function and may include, but are not limited to, the following: (i) fixation devices such as bone plates, bone screws (including compression, lag and interference screws), nails (including intramedullary nails), pins, staples, sutures, (ii) external fixation devices, (iii) components of reconstructive implants (including components of hip, knee elbow and shoulder joint replacements), (iv) adhesives and bone cements, (v) bone void fillers, and (vi) devices that are used in the repair and remodeling of soft tissue (i.e., ligament, tendon, muscle, cartilage, or other soft or connective tissue), such as an anchor, a suture anchor, a screw, and a wedge. This list is by no means considered to be exhaustive.

Both non-bioresorbable and bioresorbable materials can be used to deliver the CaSR agonist and the calcium salt to the subject. A bioresorbable (or biodegradable or bioerodible) material is defined as a material which starts to degrade upon exposure to physiological conditions and is resorbed by the body to be slowly replaced by advancing tissue (such as bone). In general, bioresorption is either by enzymatic degradation or exposure to water in vivo, by surface or bulk erosion. Bioresorbable polymeric matrices are preferred. Such polymers may be natural or synthetic polymers. Synthetic polymers are preferred as they are immunologically inert, that is they do not initiate an immune response in a subject. One criterion for the selection of the polymer is the period of time over which release is desired, generally in the order of a few hours to a year or longer. Typically, release over a period ranging from between a few hours and three to twelve months is most desirable. A further criterion for selection of the polymer is its mechanical properties, this is particularly important in the case of medical devices that may be load-bearing.

The CaSR agonist and the calcium salt can be delivered using a bioresorbable device by way of diffusion, or by degradation of the polymeric matrix. Exemplary synthetic polymers which can be used to form at least part of the device include, but are not limited to: polyamides, polycarbonates, polyalkylenes, polyalkylene glycols, polyalkylene oxides, polyalkylene terepthalates, polyvinyl alcohols, polyvinyl esters, polyvinyl esters, polyvinyl halides, polyvinylpyrrolidone, polyglycolides, polysilaxanes, polyurethanes and co-polymers thereof, alkyl cellulose, hydroxylalkyl celluloses, cellulose ethers, cellulose esters, nitro celluloses, polymers of acrylic and methacrylic esters, methyl cellulose, ethyl cellulose, hydroxypropyl cellulose, hydroxyl-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butamethylacrylate), poly(isobutylmethacrylate), poly(hexylmethacrylate), poly(isodecyl methacrylate), poly(laurylmethacrylate), poly(phenylmethacrylate), poly(methyl acrylate), poly(isopropyl acrylate), poly(isobutyl acrylate), poly(octadecyl acrylate), poly(ethylene glycol), poly(ethylene oxide), poly(ethylene terephthalate), poly(vinyl alcohols), polyvinyl acetate, polyvinyl chloride, polyvinylpyrrolidone, and polymers of lactic acid and glycolic acid, polyanhydrides, poly(ortho)esters, poly(butyric acid), poly(valeric acid), and poly(lactide-co-caprolactone) and natural polymers such as alginate and other polysaccharides including dextran and cellulose, collagen, chemical derivatives thereof (substitutions, additions of chemical groups), for example, alkyl, alkylene, hydroxylations, oxidations, and other modification routinely made by those skilled in the art, albumin and other hydrophilic proteins, zein, and other prolamines and hydrophobic proteins, copolymers and mixtures thereof.

Examples of non-resorbable polymers include polyethylene, polypropylene, polystyrene, ethylene vinyl acetate, poly(methyl)acrylic acid, polyamides, copolymers and mixtures thereof.

Bioadhesive polymers that are suitable for the present invention include hydrogels, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate) poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate).

A particular bioresorbable polymer suitable for use in the present disclosure is poly(DL-lactide-co-glycolide (PDLG) (85:15).

In embodiments of the disclosure, at least part of a surface of the medical device is coated or impregnated with a material comprising the CaSR agonist and the one calcium salt. For example, it is envisaged that the material is a bioresorbable polymeric matrix which is provided as a surface layer, for example, as a coating or film, on the surface of a medical device. As the polymeric component is resorbed, the CaSR agonist and the calcium salt are released. As an example, an acetabular cup component of a hip implant can be provided with a surface coating having both osteoconductive properties, promoting bony ingrowth into at least part of the surface of the implant and also osteogenic properties. This osteogenesis can be associated with the release of a calcimimetic from the surface layer. For example, at least part of the surface layer can comprise a bioresorbable polymeric component in admixture with a calcimimetic, such that as the polymeric component is resorbed over time the calcimimetic is released and increases/enhances the sensitivity of CaSR. In further embodiments of the invention this further component also comprises at least one calcium salt can be transiently released.

Alternatively the device includes a material comprising the CaSR agonist and the calcium salt. For example, an osteoconductive interference screw, which is used in anterior cruciate ligament (ACL) repair, including poly-DL-lactide glycolide (85:15) in admixture with at least one CaSR agonist and at least one calcium salt. In further embodiments of the disclosure, the interference screw includes poly-DL-lactide glycolide (85:15) in admixture with an amino acid and a calcium salt, wherein the amino acid is an aromatic acid such as phenylalanine (Phe), tyrosine (Tyr) and tryptophan (Trp) or aliphatic amino acid such as glycine (Gly), alanine (Ala), valine (Val), leucine (Leu) and isoleucine (Ile) and the calcium salt is Cl₂ or CaCO₃ In further embodiments of the disclosure, the amino acid is in the L-stereoisomeric form.

In embodiments of the disclosure, wherein the CaSR agonist and the calcium salt are releasable from a polymeric component of the device, this polymeric component is provided at a concentration of less than about 1% or between about 1-5%, 6-10%, 11-15%, 16-20%, 21-25%, 26-30%, 31-35%, 36-40%, 41-50%, 51-60%, 61-65%, 66-70%, 71-75%, 76-80%, 81-85%, 86-90%, 91-95% or greater than about 95% w/w of the device itself or the coating of the device. The calcium salt is provided at a concentration of less than about 1% or between about 1-5%, 6-10%, 11-15%, 16-20%, 21-25%, 26-30%, 31-35%, 36-40%, 41-50%, 51-60%, 61-65%, 66-70%, 71-75%, 76-80%, 81-85%, 86-90%, 91-95% or greater than about 95% w/w of the device itself or the coating of the device. The CaSR agonist is provided at a concentration of less than about 1% or between about 1-5%, 6-10%, 11-15%, 16-20%, 21-25%, 26-30%, 31-35%, 36-40%, 41-50%, 51-60%, 61-65%, 66-70%, 71-75%, 76-80%, 81-85%, 86-90%, 91-95% or greater than about 95% w/w of the device itself or the coating of the device.

In a specific embodiment of the disclosure, the device comprises a polymeric component which is between about 1% to about 30% w/w of the device or coating, a calcium salt which is about 1% to about 50% w/w of the device or coating and a CaSR agonist which is about 1% to about 20% w/w of the device or coating.

In a further specific embodiment of the disclosure, the device comprises a polymeric component, this component itself including poly(DL-lactide-co-glycolide (PDLG) (85:15), and being about 1% to about 30% w/w of the device or coating, the device further including calcium carbonate which constitutes about 1% to about 50% w/w of the device or coating and an L-amino acid which constitutes about 1% to about 20% w/w of the device or coating.

According to a further aspect of the disclosure, there is provided a method of enhancing the sensitivity of the CaSR to Ca²⁺ in a subject, the method comprising the step of implanting a device comprising at least one CaSR agonist and at least one releasable calcium salt into a subject.

In embodiments of the disclosure, the CaSR agonist is a type I agonist such as an inorganic polycation, an organic polycation, an aminoglycoside antibiotic or an arylalkyl polyamine. In alternative embodiments of the disclosure, the agonist is a type II agonist such as an aromatic or aliphatic amino acid selected from the group consisting of Phe, Tyr, Trp, Gly, Ala, Val, Leu or Ile. In particular, the amino acid is in the L-stereoisomeric form.

In still further embodiments of the disclosure, it is envisaged that the device comprises at least one type 1 agonist and at least one type II agonist.

In embodiments of the disclosure, it is envisaged that the device comprises two or more different type 1 agonists. For example, the device can comprise two or more different polyvalent cations (e.g Mg²+ and Zn2+). In other examples the device can comprise a polyvalent cation (e.g Mg²⁺) and an aminoglycoside antibiotic (e.g neomycin).

In further embodiments of the disclosure, it is envisaged that the device comprises two or more different type 2 agonists, for example an aromatic amino acid (e.g L-Phe) and aliphatic amino acid (e.g L-Gly).

In still further embodiments of the disclosure, it is envisaged that the device comprises at least one type 1 agonist and at least one type II agonist.

Suitable Ca²⁺ salts include, but are not limited to, calcium carbonate (CaCo₃), calcium chloride (CaCl₂), calcium phosphate, and calcium sulfate.

In some embodiments of the disclosure, the mode of release of the calcium salt is such that a therapeutic concentration of calcium salt of between about 0.5 mM and 5 mM is provided in the vicinity of the device, more particularly a concentration of between about 1 mM and 3 mM and even more particularly a concentration of between about 1 mM and 2.5 mM.

In advantageous embodiments of the disclosure, the releasable L-amino acid is L-Phe and the releasable calcium salt is CaCl₂, wherein the therapeutic concentration of L-Phe in the vicinity of the device is between about 0.5 mM and 5 mM, more particularly between about 1 mM and 3 mM, and even more particularly between about 1 mM and 2.5 mM, and wherein the therapeutic concentration of CaCl₂ in the vicinity of the device is between about 0.5 mM and 5 mM, more particularly between about 1 mM and 3 mM, and even more between about 1 mM and 2.5 mM.

In a particularly advantageous embodiment of the disclosure, the releasable CaSR agonist is L-Phe, providing a therapeutic concentration in the vicinity of the device of about 2.5 mM and the releasable calcium salt is CaCl₂, providing a therapeutic concentration in the vicinity of the device of about 2.5 mM.

In further advantageous embodiments of the disclosure, the releasable L-amino acid is L-Trp and the releasable calcium salt is CaCl₂, wherein the therapeutic concentration of L-Trp in the vicinity of the device is between about 0.5 mM and 5 mM, more particularly between about 1 mM and 3 mM, and even more particularly between about 1 mM and 2.5 mM, and wherein the therapeutic concentration of CaCl₂ in the vicinity of the device is between about 0.5 mM and 5 mM, more particularly between about 1 mM and 3 mM, and even more between about 1 mM and 2.5 mM.

In embodiments of the method, the device is implanted at or near a site at which the controlled release of at least one CaSR agonist and at least one calcium salt would be therapeutically beneficial. The device can be a medical device or a surgical device.

The device includes a bone plate, a bone screw, a compression screw, a lag screw, an interference screw, a nail, an intramedullary nail, a pin, a staple, a suture, an external fixation device, a reconstructive joint implant, a component of a hip, knee, elbow or shoulder implant, an adhesive, a bone cement or a bone void filler, or a soft tissue fixation device, such as an anchor, a suture anchor, a screw, or a wedge. These devices are implanted at or near at least one of a bone, a tendon, a ligament, or a combination thereof.

The method can be used to promote or enhance ossification at a specific site within a subject, by providing the device at or near the site. Ossification is defined as the hardening or calcification of soft tissue into a bonelike material.

It is further envisaged that in some situations more than one device may be implanted into the subject. When more than one device is implanted into the subject, the devices can be the same or different. For example, in a total hip replacement procedure the devices can include the acetabular cup, femoral head and stem and cement.

According to a further aspect of the disclosure, there is provided the use of a device according to the disclosure to promote ossification in a subject.

In yet a further aspect of the disclosure, there is provided a composition comprising a polymer material, a calcium sensing receptor agonist, and a calcium salt.

### BRIEF DESCRIPTION OF THE DRAWINGS

FIG. 1 shows Alizarin red staining of mouse calvarial cells cultured for 2 weeks in media with (a) 2.5 mM CaCl₂; (b) 2.5 mM CaCl₂ and 2.5 mM L-tryptophan; (c) 2.5 mM CaCl₂ and 2.5 mM L-phenylalanine.

FIG. 2 shows spectrophotometric quantification of Alizarin red staining illustrated in FIG. 1.

FIG. 3 shows Von Kossa staining of mouse calvarial cells cultured for 4 weeks in media with (a) 2.5 mM CaCl₂ and (b) 2.5 mM CaCl₂ and 2.5 mM L-phenylalanine.

FIG. 4 shows quantification using image analysis of Von Kossa staining from mouse calvarial cells cultured for 2 weeks with (a) 2.5 mM CaCl₂ or (b) 2.5 mM CaCl₂ and 2.5 mM L-phenylalanine.

FIG. 5 shows tendon-bone repair strength using suture anchors of the present disclosure.

### DETAILED DESCRIPTION

### Example One

Osteoblastic cells were isolated from mouse calvarial tissue dissected from the heads of 2-3 day old freshly slaughtered mice pups using sequential enzymatic digestion. Cells were maintained in DMEM+10% Foetal Bovine Serum (FBS) at 37° C./5% CO₂ in order to expand the cells before experimental use. Human dermal fibroblasts (HDFs) were used in experiments as an additional negative control.

Cells were seeded into 24 well plates at a concentration of 5×10⁴ cells/well in αMEM+10% FBS (n=6). Experiments were set up to examine the amount of mineralisation at two different concentrations of calcium chloride (CaCl₂) (1.8 mM and 2.5 mM) with and without L-amino acids (L-phenylalanine and L-tryptophan at 2.5 mM). Cells were cultured at 37° C./5% CO₂ with media changes three times a week. After 2 weeks and 3 weeks, the monolayers were stained to show the extent of mineralisation using Alizarin red (calcium deposition) and after 4 weeks with Von Kossa (mineralised matrix). Alizarin red stained monolayers were destained and the stain quantified spectophotometrically, whilst the Von Kossa staining was quantified using image analysis.

Alizarin red staining of the HDF cells showed there to be no mineralisation occurring in these cultures (images not shown), which is as expected as these cells do not have the ability to mineralise matrix and demonstrates that the staining is positive for calcium deposition.

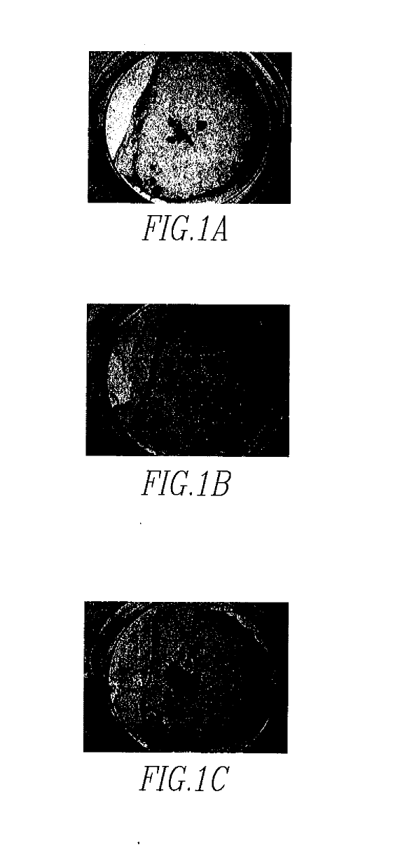

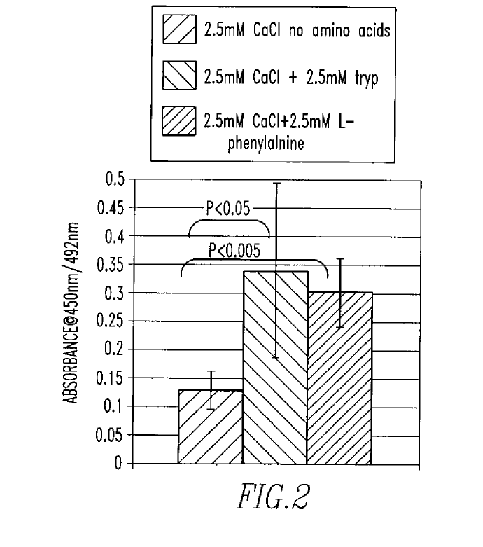

As illustrated in FIG. 1 and FIG. 2, the presence of L-Trp or L-Phe at a concentration of 2.5 mM significantly increases calcium deposition in mouse calvarial cells over a 2 week period when compared to 2.5 mM CaCl₂ alone.

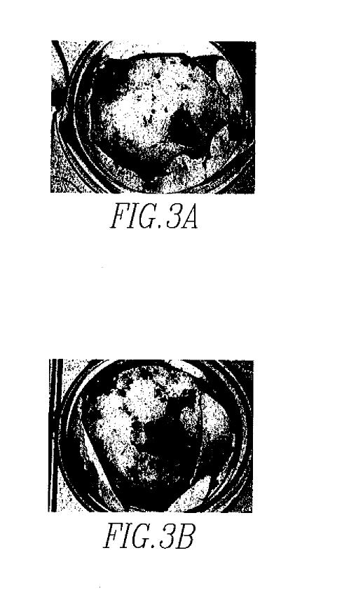

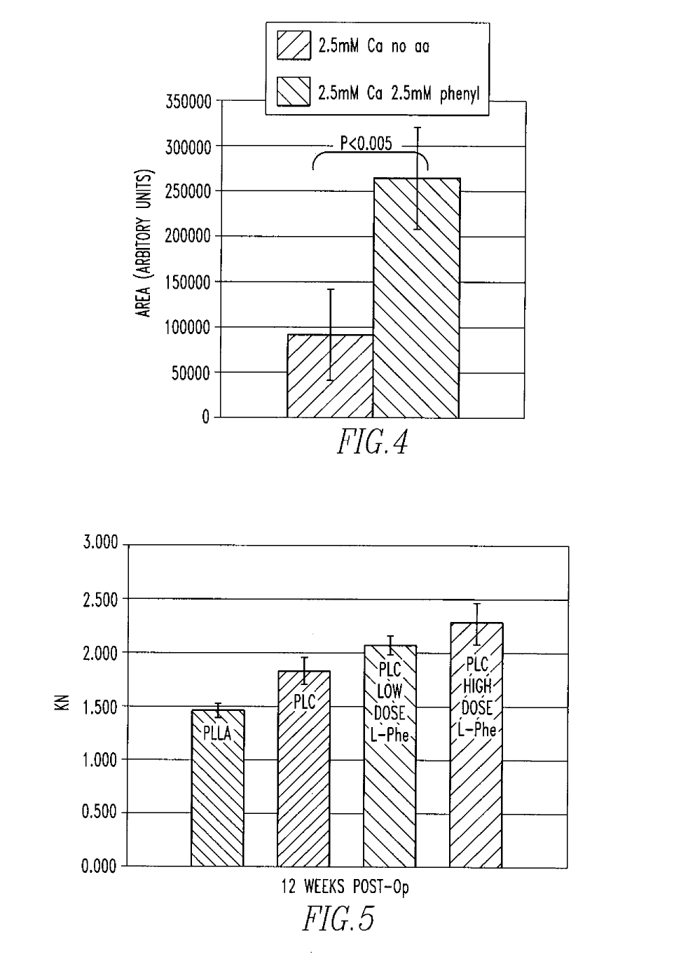

As illustrated in FIG. 3 and FIG. 4 the presence of L-Phe at a concentration of 2.5 mM significantly increases the amount of mineralised matrix in mouse calvarial cells over a 4 or 2 week period, respectively, when compared to 2.5 mM CaCl₂ alone.

For FIGS. 2 and 4, means are +/− s.d. and statistics is one-way ANOVA with turkeys pair comparison.

### Example Two

Female Cross-bred sheep were used to evaluate suture anchors comprised of different materials to re-attach a patellar tendon to the tibial tuberosity. A 3-4 cm transverse skin incision was made over the insertion site of the patella tendon to the tibial tuberosity. The tendon was then carefully pared off the bone. Using a standard template, two 3.0 mm holes are drilled in the prepared bone bed, medially and laterally and a suture anchor was inserted into each hole and the suture passed vertically through the tendon. The anchor group details are shown below.

Group 1. Poly L Lactide (PLLA) suture anchor. Inert bioabsorbable anchor

Group 2. Poly dl lactide-co-glycolide with calcium carbonate (35% w/w) (PLC).

Group 3. Poly dl lactide-co-glycolide with calcium carbonate (34% w/w) and phenylalanine (1% w/w) (PLC+low dose L-Phe).

Group 4. Poly dl lactide-co-glycolide with calcium carbonate and phenylalanine high dose (3.5% w/w) (PLC+high dose L-Phe)

At 12 weeks the femurs were resected and the patella tendon was cleared of all other tissue, thereby leaving the patella joined to an intact tibia by the tendon. The interface between the tibia and the tendon was mechanically tested to failure. The intact tibia was clamped down in a drilling jig and two 6.0 mm holes drilled through the bone. The bone was then placed in a test jig with two pins through the jig and bone. The patella was placed in a ring mould, which was filled with PMMA cement. Upon hardening of the cement, the ring mould was removed.

The jig was attached to the cross head of an Instron machine, and the patella to a tension load cell. Tension was applied by movement of the crosshead at 50 mm/min to the specimen and the load recorded at failure.

On macroscopic examination there was no evidence of adverse reactions to either type of suture anchor. There was no bone resorption nor bone cysts around them nor any aggressive inflammatory tissue. The results of the mechanical testing are shown in FIG. 1. These results demonstrate the addition of a calcium releasing component to a suture anchor stimulates repair (PLC vs PLLA) of a soft tissue back to bone. The addition of a calcium sensing receptor agonist further enhances the repair strength, increasing repair strength with increasing dose of the agonist. The anchor with a high dose of phenylalanine and calcium carbonate had improved the strength of the tendon to bone repair by 56% over the inert anchor (PLLA).

We claim:

1. A device comprising a releasable calcium sensing receptor (CaSR) agonist and a releasable calcium salt.

2. The device according to claim 1, wherein the agonist is a type I agonist.

3. The device according to claim 2, wherein the type 1 agonist is an inorganic polycation, an organic polycation, an aminoglycoside antibiotic or an arylalkyl polyamine.

4. The device according to claim 1, wherein the agonist is a type II agonist.

5. The device according to claim 4, wherein the type II agonist is an aromatic or aliphatic amino acid selected from the group consisting of Phe, Tyr, Trp, Gly, Ala, Val, Leu and Ile.

6. The device according to claim 5, wherein the amino acid is the L-stereoisomer.

7. The device of claim 1, wherein the calcium salt is selected from a group consisting of calcium carbonate, calcium chloride, calcium phosphate, and calcium sulphate.

8. The device according to claim 1, wherein the device is selected from a group consisting essentially of a bone plate, a bone screw, a compression screw, a lag screw, an interference screw, a nail, an intramedullary nail, a pin, a staple, a suture, an external fixation device, a reconstructive joint implant, a component of a hip, knee, elbow or shoulder implant, an adhesive, a bone cement, a bone void filler, and a soft tissue fixation device.

9. The device according to claim 8, wherein the soft tissue fixation device is a suture anchor.

10. The device according to claim 1, wherein the device is at least partially bioresorbable and wherein the CaSR agonist and the calcium salt are released from the medical device as it is resorbed.

11. A method of enhancing the sensitivity of the CaSR to Ca²⁺ in a subject, said method comprising providing a medical device according to claim 1.

12. The method according to claim 11, wherein the medical device is implanted at or near at least one of a bone, a tendon, a ligament or a combination thereof.

13. The method according to claim 10, wherein the medical device is selected from a group consisting essentially of a bone plate, a bone screw, a compression screw, a lag screw, an interference screw, a nail, an intramedullary nail, a pin, a staple, a suture, an external fixation device, a reconstructive joint implant, a component of a hip, knee, elbow or shoulder implant, an adhesive, a bone cement or a bone void filler, and a soft tissue fixation device.

14. The method according to claim 13, wherein the soft tissue fixation device is a suture anchor.

15. The method according to claim 11, wherein the enhanced sensitivity of the CaSR to Ca²⁺ promotes ossification.

16. A method of using a device according to claim 1 to promote ossification in a subject.

17. A composition comprising a polymer material, a calcium sensing receptor agonist, and a calcium salt.
